(19) United States
(12) Patent Application Publication
(10) Pub. No.: US 2001/0049101 A1
(43) Pub. Date: Dec. 6, 2001

(54) Micro-label biological assay system

(76) Inventors: Brian Brogger, Andover, MN (US); Justin Esterberg, Andover, MN (US); William Kerns, New Brighton, MN (US)

Correspondence Address: Beck and Tysver, Suite 100, 2900 Thomas Ave. S., Minneapolis, MN, 55416, US

(21) Appl. No.: 09/790,285

(22) Filed: Feb. 22, 2001

Related U.S. Application Data
(60) Provisional application No. 60/193,793, filed on Mar. 31, 2000.
(60) Provisional application No. 60/184,295, filed on Feb. 23, 2000.
(60) Provisional application No. 60/184,426, filed on Feb. 23, 2000.
(60) Provisional application No. 60/213,814, filed on Jun. 23, 2000.

(51) Int. Cl. C12Q 1/68

(52) U.S. Cl. 435/6

(57) ABSTRACT

A small micro-label with a machine-readable indicia is used to react with and identify analytes in a multiplex reaction with biologic molecules.

### CROSS REFERENCE TO RELATED APPLICATIONS

The present application claims the benefit of the following provisional applications; Ser. No. 60/193,793 filed Mar.31, 2000 entitled “Bio-Taggants”; 60/184,295 filed Feb. 23, 2000 entitled “Biologic Assay Identification Labels”; Ser. No. 60/184,426 filed Feb. 23, 2000 entitled “Biologic Assay Micro-Labels”; Ser. No. 60/213,814 filed Jun. 23, 2000 entitled “Microfiche Microetch Biotaggant”. Each of these applications is incorporated by reference in its entirety herein.

### FIELD OF INVENTION

The present invention relates generally to the detection and identification of biological molecules and reactions. More particularly, it relates to the use of microscopic particulates for the retrospective identification of specific reactions with analytes.

2. Background of the Invention

The Human Genome Project as well other gene sequencing and drug discovery projects have intensified the development of products that react with and detect biologic molecules such as DNA. The most commercially significant method for multiplex DNA analysis is the two-dimensional microarray, or “gene-chip”. In this analytical platform, probe DNA is arranged in an X-Y coordinate system where the identity of the particular probe is encoded by its physical position on the array. Typically a photolithographic masking process is used to synthesize oligonucleotides (short segments of DNA) directly onto silicon wafers in a specific X-Y coordinate pattern. These microarrays excel at high-density gene sequencing and differential expression applications. However, the microarrays are very expensive to produce since they require a time intensive photolithographic process that involves the sequential production of many photomasks to synthesize the oligonucleotide sequences. A related technology is the so-called “spotted array” in this system the probe DNA is pipette onto small spots on a substrate. Once again retrospective identification relies on the X-y placement of the spots.

More recently, a multiplex test has been developed based on “carrier” technologies. In these systems, small microspheres are uniquely coded and probes of interest are chemically attached to the microspheres using standard protocols. The microspheres are then combined into a multiplex test. Generally, these carrier-based technologies use microspheres diameter or color to retrospectively identify the reaction. These microspheres typically have diameters on the order of 5 microns. Two such technologies are under development by Luminex, Austin, Tex. USA, and Illumina, San Diego, Calif. USA.

In the Luminex system, a conventional flow cytometer is used to detect and Ser.ly analyze the microspheres. However, in the Luminex system each microsphere passes though the optical reading system Serially and just once. As a consequence the amount of time available to measure a microsphere is limited.

Another limitation of the technology is the fact that the total No. of distinguishable characteristics such as size and color are small, thus limiting the size of the multiplex test. There are also difficulties in interpreting the identity of the microspheres because multiple fluorophores are used to identify the spheres are also used to quantify the magnitude of the chemical reactions on the microspheres. Also the broadband florescence of the dyes makes it difficult measure light intensities at different wavelengths quickly and unambiguously. An additional drawback is the requirement to calibrate each batch of microspheres since there are inter-batch differences in microsphere diameter.

The Illumina system involves the use of a fiberoptic microwell system to read microspheres. Once the microspheres are reacted in the test and deposited at the ends of a multi-fiberoptic channel cable they are immobile. The microspheres are collected so that one sphere fits in each microwell. The fiberoptic is used to pass light used to identify the microsphere code and its reaction. Here the ability to use a large No. of microspheres is compromised by the requirement that they be readily distinguishable in the microwell.

For these reasons among others it is desirable to develop improved systems which allow the retrospective idnetification of reactions occurring on small particles.

### SUMMARY OF THE INVENTION

In contrast to the prior art, this invention involves the use of an optically coded particle hereafter referred to as a “micro-label”. In use these micro-labels are coated with a reactive chemistry or linking chemistry which is used to attach a probe molecule.

Each micro-label has a unique set of characters or set of markings which can be “read” or identified from either side of the preferred transparent micro-label in either white light or single frequency laser light. Both alphanumeric and barcodes are illustrative examples of suitable marking techniques. The preferred encoding process is o alter the dept or thickness of the particle at the indicia. The variations in optical path length gives rise to readable contrast in a No. of optical readers.

The readability of the preferred optical path length indicia does not depend on “color coding” of the micro-label. The contrast between the background and the marking is detectable over a very broad range of reading wavelengths. In this sense the micro-labels indicia is not spectrally dependent. The label will typically be read “automatically” with optical recognition equipment but the micro-labels are distinguishable by the human eye with the aid of magnification as well. The preferred reader is an automated camera/microscope system, which is widely available. The microscope will be used with conventional pattern and optical character recognition software. The identity of the micro-label can be read with bright field, dark field or phase contrast illumination.

In a preferred method of use a mixed set of micro-labels with appropriate surface chemistry and probe molecules are combined and can be used to analyze a single solution for multiple analytes. This is called a “multiplex test”. In general the whole surface of the micro-label is used to react with the probe and analyte chemistry, however it may be desirable to limit the active area to a fixed area on the micro-label. One method of manufacture is to directly write the indicia with an eximer laser onto the polymer substrate. The laser may be used to activate or inactivate regions of the label to minimize non-specific binding and the like. This surface modification process is optional.

Generally, the micro-labels are thin and tend to lie flat on a reading substrate. Specific molecular probes such as DNA, proteins, haptens or other molecules or biological particles can be chemically attached to each unique micro-label. The unique indicia or coding on the micro-label is then used for retrospective identification of the reaction with the attached probe.

The identification of the presence of analytes can be performed using one or more so called reporter molecules attached to the analytes. Appropriate reporter molecules include those with distinguishable optical spectra characteristics. Candidate reporter molecules may be taken from the families of organic dye molecules including colorimetric, fluorometric, or other spectrophotometric reporter molecules. A preferred reporter molecule is the so called “quantum dot”. These semiconductor nanocrystal are luminescent over a very narrow bandwidth and more fully described in U.S. Pat. 5,990,479 which is incorporated by reference herein.

The micro-labels described herein can be “read” in an examination zone while stationary or while in motion. Consequently the examination zone may include, but not be limited to, flat glass slides, etched glass slides, flow-based systems, and microwell/microtiter plates. The micro-label identity and presence of biochemical reaction at its surface can be analyzed qualitatively and semi-quantitatively, or quantitative analysis can be performed in the examination zone.

The analysis requires that the identity of the micro-label and the presence and extent of biochemical reactions on the surface be determined. The instrument utilized for micro-label analysis must have the ability to read and identify the micro-label indicia and its respective reporter molecule(s). It is preferred to have the identity and reporter molecule reaction read essentially at the same time and preferably when illuminated in white light.

The multiplex assays or tests designed utilizing these micro-labels as a basis can be used to test for a variety of molecules or biological particles. Nucleic acid based molecules such as DNA, RNA, and single nucleotide polymorphisms (SNPs) can be tested. Proteins such as antibodies, antigens, haptens, transcription/translation factors, enzymes, membrane proteins, glycoproteins, can also be tested. Other biochemicals such as hormones, cytokines, neurotransmitters, neuromodulators, pharmaceuticals are also examples.

Types of analyses include expression profiling, differential expression, genetic sequencing, protein sequencing, and biomolecular structural and function analysis. The general fields of application of such tests include life science research, biomedical research, clinical in vitro diagnostic (IVD) tests, pharmaceutical design/development, pharmacogenomics, genomics, and proteomics.

### BRIEF DESCRIPTION OF THE DRAWINGS

An exemplary version of a micro-label and system and method for retrospectively identifying chemical and biological reactions in the is shown in the figures, wherein like reference numerals refer to equivalent structure throughout, and wherein:

FIG. 1 is a schematic diagram showing the use of the micro-labels;

FIG. 2 is a schematic diagram showing the use of the micro-labels;

FIG. 3 is a schematic diagram showing the reading of a micro-label;

FIG. 4 is a schematic diagram showing an alternate use of the micro-labels;

FIG. 5 is a schematic diagram showing an alternate use of the micro-labels;

FIG. 7 is a schematic diagram showing an alternate use of the micro-labels;

FIG. 8 is a schematic diagram showing alternate use of the micro-labels;

FIG. 9 is a schematic diagram showing the reading of a micro-label; and,

FIG. 10 is a schematic diagram showing the preferred micro-label.

### DESCRIPTION OF THE INVENTION

Nomenclature

The particles are called “micro-labels” each micro-labels has an “indicia” on it to identify the micro-label. The micro-labels have large biological molecules attached to them, which are referred to as reaction “probes”. These molecules are reacted with complimentary molecules that are referred to as “analytes”. The competitive binding or reaction with these sites is referred to as “hybridization”. The process of identifying the identity of the micro-label is called “reading” the identity of the micro-label. Each analyte will typically have a so called “reporter molecule” attached to it. In the reaction the reporter molecules quantify the reaction in general the more reporter molecules attached to he micro-label the greater the magnitude of the reaction. The reader functions to count photons from the reporter molecules to quantify or read the reaction in the “reader”. In the figures complex molecules are given a geometric interpretation to facilitate description.

Overview

In this invention micro-labels with an indicia or marking are provided with an attachment chemistry or linking chemistry which is used to bind reactive probes to the micro-labels. Preferably each micro-label has a unique set of characters or set of markings (alphanumeric or barcode) which can be “read” or identified from either side of the preferred transparent micro-label. The preferred indicia are recessed into the surface of the micro-label and the difference in optical path length between the indicia and the surrounding label give rise to contrast in the reader.

The micro-label will typically be read “automatically” but the micro-labels are distinguishable by the human eye with the aid of magnification as well. The preferred reader is an automated camera/microscope system, which is widely available. The microscope will be used with pattern and optical character recognition software which recognises the indicia.

Generally, the micro-labels are thin and tend to lie flat on a static reading substrate in a reader examination zone. The micro-labels may be read or they may be in motion.

Specific molecular probes such as DNA, proteins, haptens or other molecules or biological particles can be chemically attached to each unique micro-label. For ease of description the whole surface is illustrated as the reactive surface. It is expected that the laser etch or marking process can be used to preferentially define reactive sites to minimize non-specific binding of analyte to the micro-label.

Representative Construction of the Micro-labels

In general thousands of labels with the same unique code are manufactured at a time. It is preferred to use a polystyrene film which is passes under an eximer laser stage. The stage moves the film past the laser and the laser creates multiple recessed 2D barcodes on the film. Experimental quantities of micro-labels have been cut to size with an eximer laser. But it is anticipated that a die stamping operation will make adequate labels in production quantities.

Experiments suggest that marked and cut polystyrene film should be compressed between two pieces of film that carry the micro-labels. This laminate structure aids in the collection and handling of the micro-labels after they are cut from the film.

Typically the eximer laser will ablate the film and create a “perfect” 90-degree edge and each dot making up the bar code will have the same depth. The eximer laser results in a very “clean” cut which is desirable.

Once manufactured, the micro-labels are washed and most subsequent handling will be with a fluid carrier.

Representative Attachment Chemistry

Generally, the micro-labels are made from a polystyrene film but other polymers are acceptable as well. The polymer used for biomolecular attachment must be receptive to further modification. A common method of attachment is to carboxylate the polymer by adding monomers in the polymerization such as acrylate or methacrylate, or oxidizing polymeric particles with oxidizing agents. The carboxylate functional groups can then be reacted with other linker molecules that will bind directly to the probe biomolecules or other chemicals.

There are many other chemistries and combinations that can be used for the same purpose as carboxylate-modified surfaces. These include the use of hydrazides, maleimides, avidin, and streptavidin surfaces.

### EXAMPLE

This protocol describes a suitable procedure for an illustrative embodiment of the invention. This general procedure outlines the attachment of oligonucleotides to polystyrene labels is as follows:

1. Oxidation of polymer surfaces to impart carboxylate functionality

2. Grafting of the surface with N-methyl-1,3-propatne diamine

3. Carbodiimide attachment

4. Carbodiimide plus oligonucleotide attachment.

The oxidation of the polystyrene label surfaces was accomplished by adding approximately 1 gram of solid labels to a glass vial. This was followed by addition of approximately 2 mL 5 g KmnO₄in 0.5 M H₂SO₄(Fluka). The vials were immersed uncapped into a 60° C. water bath for 30 minutes. The oxidation mixture was then poured into separate 10 mL glass beakers. The labels were washed with 4-2 mL aliquots of 6 M HCl (5 min. each) and then 10-2 mL-aliquots of sterile water. The labels were stored at 0° C. in micro centrifuge tubes until use.

The grafting of the polymer surface was accomplished by taking about 50 of polymer labels and placing them into polypropylene micro centrifuge tubes. This was followed by addition of 400 mL 0.1 M MES, 0.1% 1-ethyl-3(3-dimethylaminopropyl)-carbodiimide (EDC), 0.001 M N-methyl-1,3-propane diamine (solution made fresh prior to use in sterile water). Reaction was allowed to proceed at 23° C. for 2 hours while shaking and intermittently vortexing the mixture. The labels were washed (350 mL and 5 minutes per aliquot) with 3 aliquots 0.1 M MES, 5 aliquots PBS, and 2 aliquots sterile water.

The oligonucleotide attachment was done by taking 5′ C6 amino-modified 20-mer poly-T (Oligos Etc., Eugene, OR) and dissolving it into solutions of sterile water at three concentrations for use in experimentation (410 ng/mL, 205 ng/mL, and 12.8 ng/mL. Depending on the condition tested, 100 mL of these solutions were added a separate sterile Eppendorf tube with 900 mL ice-cold 100 mM 1-methylimidazole pH 7, to dilute ten-fold. Then 150 mL of these diluted solutions were added to the labels followed by 50 mL 0.2 M EDC in 10 mm 1-methylimidazole pH 7 (made fresh daily). The mixture was then incubated at 50° C. for 5 hours then washed 3 rapid aliquots (350 mL) of 50° C. 0.5 M NaOH in 0.25% sodium dodecyl sulfate (SDS), 1 aliquot for 5 minutes, then 3 rapid aliquots.

Hybridization was performed by first washing the labels with 2 aliquots of 500 mL 1.5 M NaCl, 10 mM EDTA in sterile water. A solution of 500 nmol/mL of 5′ FITC-labeled 20-mer poly-A (Oligos Etc., Eugene, OR) was made 1.5 M NaCl, 10 mM EDTA. 130 mL of this solution was added to the labels, the tubes were capped with holes punched in their tops and incubated at 42° C. overnight (15 hours). Depending on the condition tested, the tubes were removed at shorter incubation times. The labels were then washed with 3 rapid aliquots (500 mL each) 0.5 M NaOH, 0.25% SDS (50° C.).

While the above description of reaction chemistry represents the preferred substrate, polystyrene, and the preferred attachment carbodiimide chemistry, it must be understood that many other substrates and reaction chemistries can be employed in the production and application of the micro-labels described herein.

There are many substrates detailed below that could be used alone or with others provided they are chemically compatible with each other and with the surface enhancement chemistry. Copolymers can be made of any two or more of these as long as they are chemically compatible. They also need to be of sufficient molecular weight and have sufficient crosslinking percentages so they will be durable through the wet chemistry used to attach biological molecules to the substrate surfaces. The substrate possibilities are not limited to those stated here.

Generally the substrates could be compose of, but not limited to, organic polymers, inorganic polymers, copolymers thereof, and crystalline solids. Specifically, polystyrene, Poly-4-vinyl-benzoic acid copolymers of polystyrene and poly-4-vinyl-benzoic acid, polyethylene, polypropylene, polyamides, nylon-66nylon-6, polyesters like polyethylene terepthalate (PET), melamine polymers, polyacrylamides, polyacrylates, polyanhydrides, maleic anyhydride-based copolymers, polymethacrylates, poly(butyl methacrylate), poly(methyl methacrylate), polycarbonates, polypeptides, polylysine, polyaspartic acid, poly(lysine-phenylalanine), hydrobromide polylactic acid, Dacron, acrylonitriles, dialdehyde, starch-methylene, dianiline, natural product polymers, polysaccharides, agarose, cellulose, nitrocellulose, glass-silica oxides, nickel oxides, aluminum oxides, titanium oxides, maganese oxide, collagen polyimides cellulose, acetate, butyrate, cellulose, triacetate, polytetrafluorethylene, poly(ethyl acrylate), poly(methyl acrylate), poly(vinyl acetate), poly(vinyl alcohol), poly(vinyl chloride), polyurethanes, polyureas, polyethers, polysiloxanes, polyphosphate, and polyphosphonate esters.

Several surface chemistry modifications can be made to make the polymers or other surface amenable to attachment of biological molecules, such as DNA, RNA, antibodies, and antigens. Generally, to covalently attach biological molecules the polymer surface must have functional (“reactive”) groups. One general method of performing this is to oxidize polymer bonds to produce carboxylate functionality to the polymer. This procedure is well referenced for polyesters and polystyrenes. This chemistry will also work well for polysaccharides and polyamides. Another possibility is to impart amine functionality to the surface. Other polymer surfaces may have hydroxyl, thiol, or amide groups exposed to impart functionality. Once the polymer substrate has functional groups it is ready for further surface modification with coupling agents. These techniques are well referenced. One common method is to utilize carbodiimides and diamines. There are numerous methods of doing this which are listed here depending on the surface functional groups: carbodiimides like 1-ethyl-3(3-dimethylaminopropyl)-carbodiimide (EDC), N,N-dicyclohexylcarbodiimide, bromocyanides, thionylchlorides, isothiocyanates, succinic anhydrides, glutaralydehydes, azides, azos, diamines, N-methyl-1,3-propane diamine, N-hydroxysuccimides N,N-dicyclohexylcarbodiimide/dioxane, triethyloxoniumtetraofluoroborate (for nylon) plus glutaraldehyde.

Another possibility to enhance attachment of probes is to directly modify them. There have been several 5′ end modifications referenced for enhancing DNA attachment. Both 5′ phosphonoates (ref), such as thiophosphate that can be reacted with p-nitrophenyl acetate, and various 5′ amines have been used. Another possibility is to use DNA that has poly-A tails that give the DNA enhanced binding characteristics. Proteins have many functional groups, such as amines, caboxylates, and hydroxyls, that give them the ability to attach to substrates.

Representative Reactive Probes

The multiplex assays designed using these micro-labels can be used to test for a variety of molecules or biological particles. Nucleic acid based molecules such as DNA, RNA, single nucleotide polymorphisms (SNPs) can be tested. Proteins such as antibodies, antigens, haptens, transcription/translation factors, enzymes, membrane proteins, glycoproteins, can also be tested. Other biochemicals such as hormones, cytokines, neurotransmitters, neuromodulators, pharmaceuticals are also examples. Types of analyses include expression profiling, differential expression, genetic sequencing, protein sequencing, and biomolecular structural and function analysis. The general fields of application of such tests include life science research, biomedical research, clinical in vitro diagnostic (IVD) tests, pharmaceutical design/development, pharmacogenomics, genomics, and proteomics. Some examples of IVD tests include tests for cardiac, liver, infectious, genetic, and neoplastic diseases. There are hundreds of currently-used IVD tests and many to be discovered, especially in the realm of genetic testing, pharmocogenomics, and cancer therapy.

Representative Analysis Apparatus

After hybridization the micro-labels are introduced into or they remain in an examination zone. There are two minimum requirements for a “reader”. First it must be able to identify the indicia on the micro-label and secondly it must be able to identify the presence of reaction on the micro-label surface (hybridization reaction between analyte and probe). To satisfy the first requirement, a CCD camera can be utilized. Digital image(s) are taken and are analyzed with software to determine the micro-label identity-and thus determine the identity of their respective probes. To satisfy the second requirement, an excitation source, and detector(s) is(are) necessary. For example, a mercury light or laser may be used to excite fluorescent response elements/reporter molecules. A detector scheme that employs a filter, and CCD camera or CCD array, etc. can be used to determine the presence and intensity of the fluorescent response. Once calibrated, the reader can also determine the concentration of analyte in the sample media.

Most traditional response elements or reporter molecules have very different absorption maxima, and may therefore require multiple excitation sources. For example, if two or three fluorescent response elements/labels with non-overlapping excitation spectra are used, to get the best response, two or three lasers tuned to their respective absorption maxima must be used. Semiconductor nanocrystals are known to have broad excitation spectra which allows one excitation source (white or UV) to be used. These nanocrystal or quantum dots are the preferred reporter molecule.

Several uniquely coded micro-labels with their respective probes may be combined into a single multiplex assay. The unique coding on the micro-label is then used for retrospective identification of the attached probe. The identification of the presence of analyte(s) can be performed using one or more of several methods including, but not limited to: calorimetric, fluorometric, or any other spectrophotometric method. Some of the labels these methods utilize include, but are not limited to: fluorophores such as those from the fluorosceine family (FITC, etc.); infrared or near-infrared fluorophores; upconverting phoshpors; fluorescing semiconductor nanocrystals of the group II-VI such as CdSe (cadmium selenide), magnesium selenide (MgSe), calcium selenide (CaSe), barium selenide (BaSe), or zinc selenide (ZnSe); or chromophores of many types. It is required that the labels or other identifiers can be detected, can be properly distinguished form one another and analyzed. The use of semiconductor nanocrystals with the micro-labels described herein may be preferred due to several benefits of these nanocrystal labels including: enhanced stability over traditional organic labels, narrower emission spectra, broader excitation spectra (ability to be excited with white or UV light), and no requirement for laser excitation.

The use of multiple colors of nanocrystals with the micro-labels as described allows for more complex testing regimens. Essentially, more information can be obtained about the analyte-sequencing, structural analysis, and functional analysis.

The micro-labels described herein can be analyzed in a variety of examination zones including, but not limited to, flat glass slides, etched glass slides, flow-based systems, and microwell/microtiter plates. The micro-label identity and presence of biochemical reaction at its surface can be analyzed qualitatively and semi-quantitatively, or quantitative analysis can be performed. The analysis in any case requires that the identity of the micro-label and the presence and extent of biochemical reactions on the surface be determined.

### DETAILED DESCRIPTION OF THE PREFERRED EMBODIMENTS OF THE INVENTION

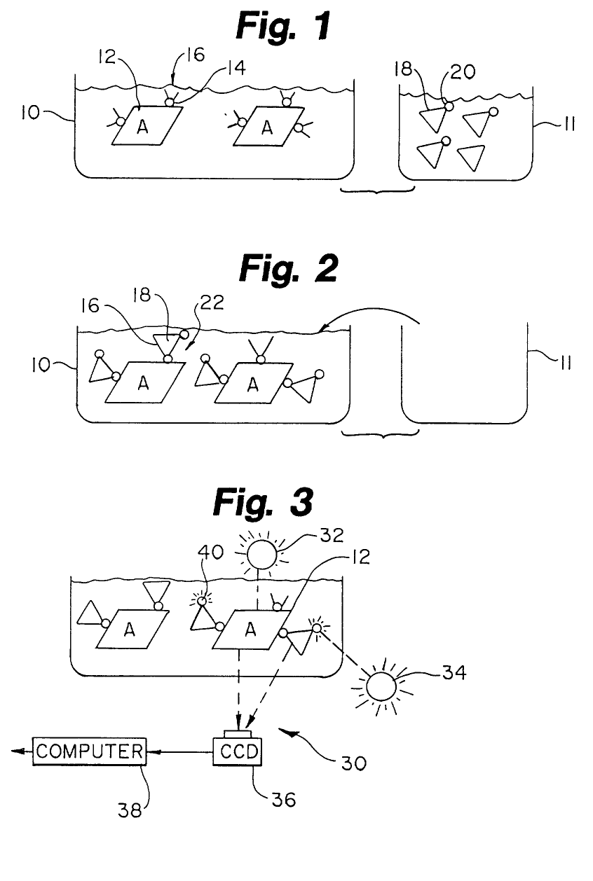

Turning the figures, FIG. 1 shows a container or well 10 with a small No. of micro-labels located within a well. Micro-label 12 has a linking chemistry illustrated as a circle at reference numeral 14. This linking chemistry is typically present all over the surface of the micro-label 12. A reactive probe molecule is illustrated as a “V” shape at reference No. 16. Analyte molecules in a separate container 11 are typified by triangular feature 18. This molecule has associated with it a reporter molecules illustrated as a circle at 20.

FIG. 2 shows typical tests where analytes from container 11 are deposited in container 10 were they react with probes on the micro-labels in well 10. The hybridization of the analytes with the micro-labels is depicted by the geometric “fit” of the analyte 18 shape with the probe 16 shape as seen at reference numeral 22. The acceptance of the “triangular probe” with the “triangular analyte” illustrates hybridization.

FIG. 3 shows the completed hybridization reaction being read with a “reader” generally designated as 30. A light source 32 is positioned to transmit light through the micro-label 12. Alternatively a light source 34 may be used to reflect light off of the micro-label 12. In either case the illuminated micro-label is positioned so that an image can be acquired by the camera 36. The image is transferred to the computer where conventional image recognition software determines the identity of the micro-label from the indicia located on the micro-label. At set forth above the preferred indicia is a 2D barcode illustrated by the alphanumeric “A” in the figure.

As seen in the figure the reporter molecules may emit energy in response to the absorption of light energy. Reporter molecule 40 is emitting energy in the figure. In general the micro-labels may be read in white light or laser light. It is a distinct advantage to use white light where possible and to use the same light source required by the reporter molecules. The preferred reporter molecule are the so-called quantum dot type nanoparticles. These emit strongly and narrowly in response to white light. In the figure the ccd camera can also measure the number of photons emitted by the reporter molecules. Generally a filter will be used to improve the signal to noise ratio of the CCD array. It is expected that pixel binning will be used to collect the total response of the micro-label.

FIG. 1, FIG. 2 and FIG. 3 taken together illustrate a simplex test where a single analyte and a single type of micro-label are used to quantify a reaction.

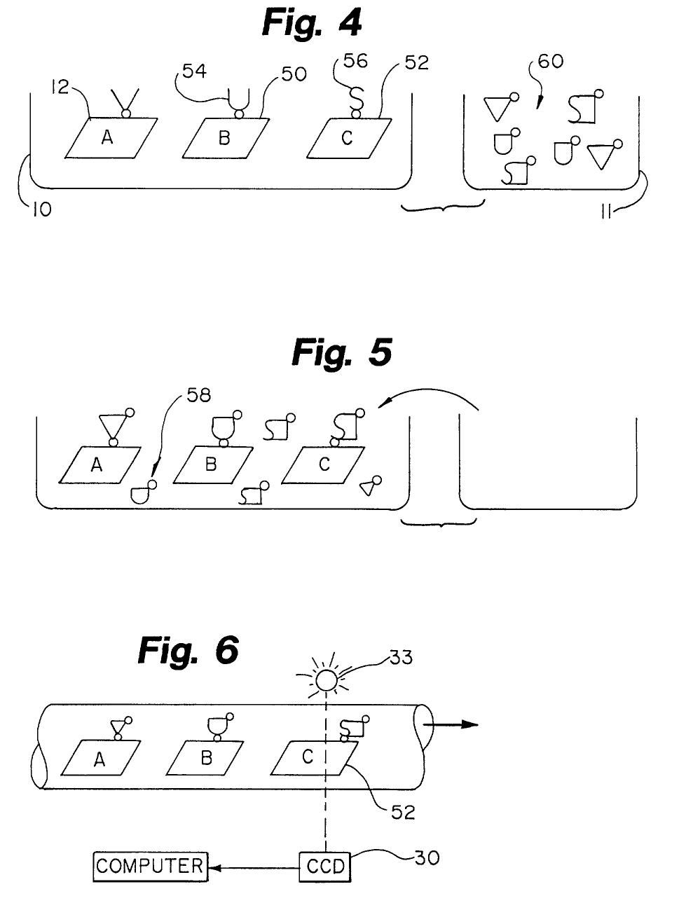

FIG. 4, FIG. 5 and FIG. 6 together show a multiplex test were three types of micro-label are pooled in a well10. Micro-label 50 carries a probe 54 and micro-label 52 carries probe 56 micro-label 12 carries a probe 16. Each probe differs and the indicia on the micro-label inform the investigator of the specific probe on each micro-label. The various analytes generally designated 60 in the test vessel 11 are reacted with the micro-labels and they bind to the appropriate sites on the micro-labels as seen in FIG. 5. The geometric interpretation of this figure is analogue to FIG. 2. The reaction is multiplex because multiple micro-labels can be used with multiple analytes to determine a broad range of reactions in a single experiment. In the reaction illustrated there are more analyte molecules than binding sites on the micro-labels which leave some analytes in solution as indicated by reference numeral 58

FIG. 6 shows the reader 30. In this embodiment the micro-labels are confined to and moved along a flow channel where they are sequentially read at the reader station. In operation the individual labels are read in a series as typified by micro-label 52 and in this embodiment the light is provided by a laser 33 for both the detection of the indicia and the detection of the reporter molecules.

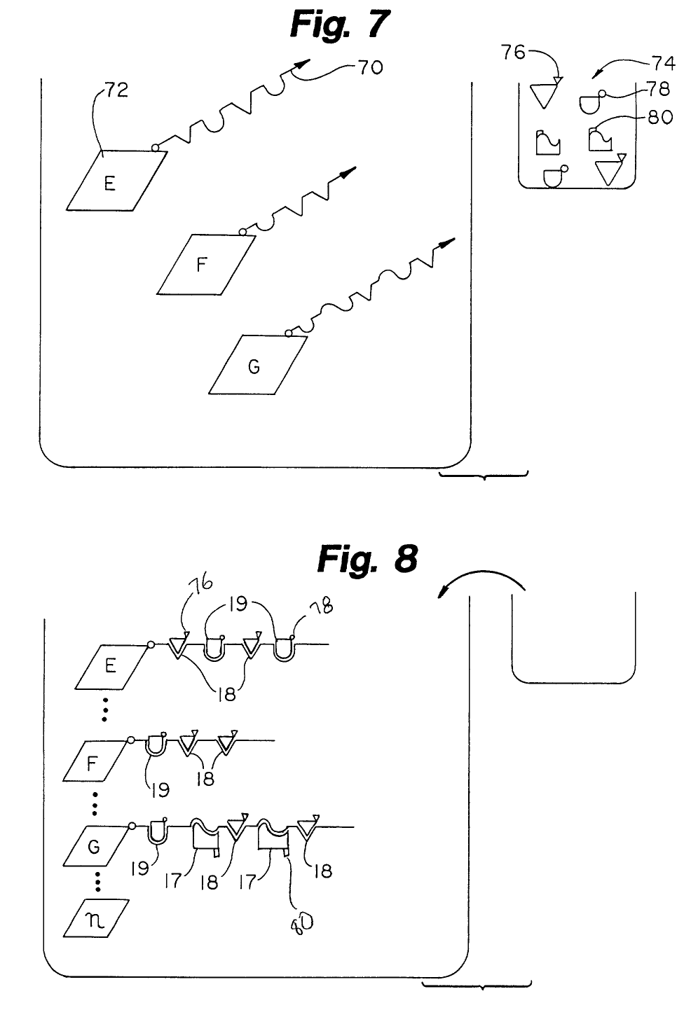

FIG. 7 shows a multifold multiplex system which attaches very long and complex probe molecules such as molecule 70 to micro-labels typified by micro-label 72. The probe 70 may have multiple binding sites for several analytes generally designated 74. By using quantum dot reporter molecules shown as reporter 76 reporter 78 and reporter 80 the narrow emission spectra allow the reader to distinguish several reporter molecules even when they occupy essentially the same space when hybridized.

FIG. 8 shows a hybridization experiment with the multifold multiplex system where individual reporter molecules such as reporter 76 reporter 78 and reporter 80 and the associated complimentary analytes shown as analyte 18 analyte 19 and analyte 17 bind with multiple sites on the various micro-labels.

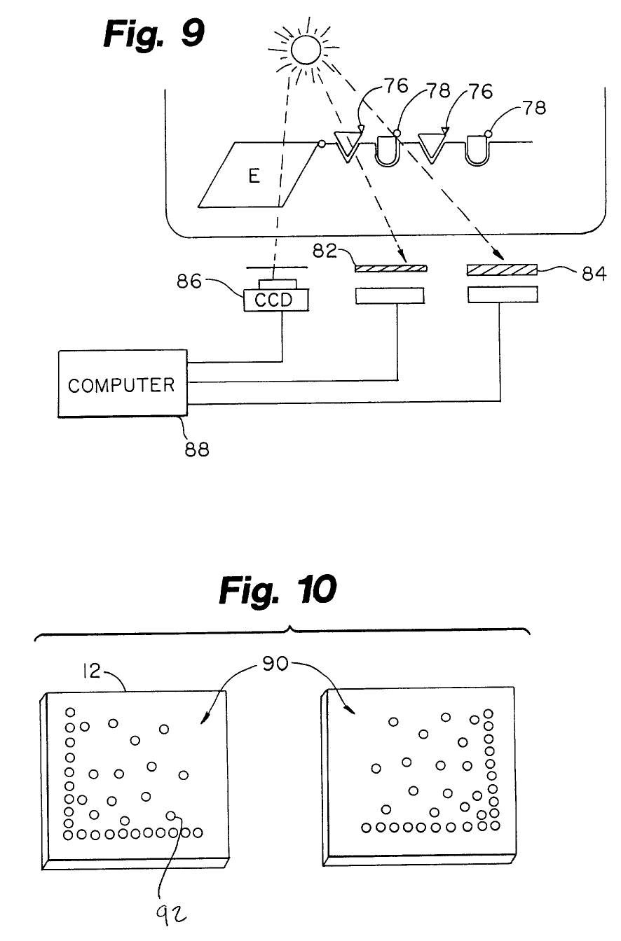

FIG. 9 shows a multispectral reading station which is the preferred system. Station 30 reads both the total response of the reporter molecules and micro-label indicia. This may be done from one image collected at a single time. It may be useful to use multiple filters 82 and 84 and mutilple cameras typified by camera 86 to collect the narrowband information form the micro-labels “all at once”.

FIG. 10 is an example of a preferred marking system of the micro-labels 12. The figure is intended to illustrate that the code or indicia generally designated 90 can be read even if the particle is “upside down”. The preferred 2-d barcode has several redundancies in it and only a partial image is required for reliable reading of the code. The transparency of the micro-labels at the observation frequencies allows the reaction to be read from both sides of the micro-label over a wide range of wavelengths. At the observation wavelengths associated with white light. In general the preferred micro-label is about 50 microns on a side and is very thin. A rectilinear and preferably square outline is preferred for the micro-label. It should be appreciated that round or circular outlines as well as irregular outline are contemplated as well. In general the indicia will be embossed or ablated into the parent material. Phase contrast or dark field observations techniques allow the indicia to be read. Other diffractive or interferometer techniques may be used as well. The difference in optical path length is caused by the laser ablation or embossing operation. In the figure the dot typified by dot 92 lies below the surface 91 of the micro-label 12. The preferred material of construction of the micro-label is polystyrene and it may be very desirable to use the laser to define a reaction zone or area on the micro-label. The surface modification can be used to reduce or eliminate non-specific binding of molecules to the micro-label thus improving the signal to noise ratio of the test.

The invention may be modified without departing from the scope of the claims.

What is claimed is:

1. A method of acquiring reaction data from analytes in a test sample comprising:
providing micro-labels with distinguishable indicia;
attaching specific reaction probes for hybridization with specific analytes of interest to specific micro-labels forming a set;
exposing and reacting said set to said test sample thereby binding analytes to probes on said micro-labels;
reading the indicia of at least one micro-label to identify the reaction probes present thereon;
reading the amount of analyte reacted with said probes on at least one micro-label to quantify the magnitude of the reaction.

2. A method of acquiring reaction data from multiple analytes in a test sample comprising:
providing micro-labels with white light visible indicia codes;
attaching specific reaction probes to specific analytes of interest to specific micro-labels forming a set;
pooling sets of said micro-labels having multiple reaction probes for multiple analytes forming a pooled set;
exposing and reacting said set of micro-labels with reaction probes to said test sample binding analytes to probes bound to micro-labels;
reading the indicia of at least one micro-label to determine the analyletes present thereon;
reading the amount of analyte reacted with said probes on at least one micro-label.

3. The method of claim 1 wherein said analytes carry a reporter molecule detectable by said analyte reading step.

4. The method of claim 2 wherein said reporter molecule is a fluorescent molecule that fluoresces after exposure to a narrow band laser light.

5. The method of claim 2 wherein said reporter molecule is a quantum dot molecule that emits a narrow band spectra after exposure to white light.

6. The method of claim 2 wherein said micro-label has a first substantially planar surface and a second substantially planar surface.

7. The method of claim 5 wherein said micro-label is transparent in white light.

8. The method of claim 6 wherein said micro-labels indicia is a 2-d barcode.

9. The method of claim 7 wherein said micro-labels are substantially rectilinear in shape and said first and second surfaces are substantially parallel to each other and the distance between said first and second surfaces is less than one half of the a rectilinear dimension of said micro-label.

10. A particle for use in a biologic assay comprising:
a planar label having a first surface and a a second surface;
an indicia located on said label.

11. The particle of claim 9 wherein said indicia is a two dimensional barcode arrayed on said first surface.

12. The particle of claim 9 wherein said indicia is etched into said first surface.

13. The particle of claim 9 wherein said indicia is embossed into said first surface.

14. The particle of claim 9 wherein said indicia is ablated into said first surface by a laser.

15. The particle of claim 9 wherein said label is made form polystyrene.

16. The particle of claim 11 wherein said polystyrene is transparent over a wide range of wavelengths.

17. The particle of claim 9 further comprising at least an active area having a linker molecule attached to said label.

18. The particle of claim 13 further comprising a probe attached to said linker molecule.

19. The particle of claim 13 wherein said area is created by exposure of the particle to laser energy to define that active area.
